(19) United States
(12) Patent Application Publication
(10) Pub. No.: US 2007/0092582 A1
(43) Pub. Date: Apr. 26, 2007

(54) Methods of preserving ophthalmic solutions and preserved ophthalmic solutions

(76) Inventor: Fu-Pao Tsao, Lawrenceville, GA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/346,013

(22) Filed: Jan. 16, 2003

Related U.S. Application Data
(60) Provisional application No. 60/350,185, filed on Jan. 18, 2002.

(51) Int. Cl. A61K 33/40; A61K 31/717; A61K 31/663

(52) U.S. Cl. 424/616; 514/57; 514/102

(57) ABSTRACT

A preservative system for aqueous ophthalmic solutions is provided, where the preservative system comprises a hydrogen peroxide source, at least one hydrogen peroxide stabilizer and an alkali earth metal salt.

The present invention relates to a method of preserving ophthalmic solutions with trace amounts of stabilized peroxy compounds and alkaline earth metal salts. U.S. Pat. Nos. 5,725,887 and 5,607,698, which are both expressly incorporated by reference herein in their entirety, disclose and claim methods for the preservation of ophthalmic solutions using stabilized hydrogen peroxide and compositions so preserved. It has now been unexpectedly discovered that the preservative efficacy of aqueous solutions preserved using stabilized hydrogen peroxide can be increased by the addition to the solutions of alkaline earth metal salts.

Trace amounts of peroxy compounds stabilized with a hydrogen peroxide stabilizer, especially diethylene triamine penta(methylene phosphonic acid) or 1-hydroxyethylidene-1,1-diphosphonic acid may be utilized as a preservative for eye wetting solutions, eye lubricating solutions, or ophthalmic active agent-containing solutions to be used in the ocular environment. Ophthalmic active agent-containing solutions contain at least one medicinal agent for application directly to the eye. The preservative according to the present invention may be used in any ophthalmic solution as long as the ingredients in that solution are compatible with trace amounts of the peroxy compounds. A hydrogen peroxide source is any peroxy compound that is hydrolyzed in water to produce hydrogen peroxide. Examples of hydrogen peroxide sources, which provide an effective resultant amount of hydrogen peroxide, include sodium perborate decahydrate, sodium peroxide and urea peroxide. It has been found that peracetic acid, an organic peroxy compound, cannot be stabilized utilizing the present system.

It is believed that most compounds, when preserved by the present invention, are compatible with trace amounts of hydrogen peroxide. Ophthalmic active agents, as used herein, are compounds that have a pharmacological effect on the eye when administered topically to the eye. The following is a non-exhaustive, non-limiting, illustrative list of ophthalmic active agents and excipients that are compatible with the preservative according to the present invention: atropine, homatropine, cyclopentolate, tropicamide, lachesine, dibutoline, oxyphenonium, eucatropine, ephedrine, carbachol, methacholine, pilocarpine hydrochloride, isoflurophate, physostigmine, neostigmine, lignocaine, cocaine, acetylcholine chloride, antazoline phosphate, betaxolol hydrochloride, demecarium bromide, dipivefin hydrochloride, erythromycin, gentamicin sulfate, homatropine hydrobromide, idoxuridine, isosorbide, lanolin, ketotifen hydrogen fumarate, naphazoline hydrochloride, neomycin sulfate, pheniramine maleate, polysorbate gelatin (Tween), pyrilamine maleate, scopolamine hydrobromide, hyaluronic acid, sodium hyaluronate, tetracaine hydrochloride, oxmetazolin, tetrahydrozoline hydrochloride, diclofenac sodium, dextran, carteolol, sulfanilamide, procaine, proparacaine hydrochloride, sulfisoxazole disolamine, indomethacin, clonidine, corynanthine, arachidonic acid, linoleic acid, inositol triphosphate, inositol phosphates, phosphatidylinositol and phosphatidylinositol phosphates.

An ophthalmic demulcent, as used herein, means a water-soluble agent, that is applied topically to the eye to protect and lubricate mucous membrane surfaces and relieve dryness and irritation. Non-limiting examples of such ophthalmic demulcents are cellulose derivatives such as carboxymethylcellulose and salts thereof, hydroxyethyl cellulose, hydroxypropyl methylcellulose, and methylcellulose; dextran 70; gelatin; polyols such as glycerin, polyethylene glycol 300, polyethylene glycol 400, polysorbate 80, and propylene glycol; polyvinyl alcohol; and povidone.

Excipients of various types compatible with the present invention include, but are not limited to polysorbate gelatin (Tween), dextrans, lanolin inositol phosphates, alkylsulfosuccinates, sulfosuccinamates, alkyl silicone sulfosuccinates, alkylpolyether carboxylates, alkylaryl polyethoxylamines, alkylarylsulfonates, alpha olefin sulfonates, alkyl sulfates, alkyl ether sulfates, alkanol amides and alkamides, alkylamphoterics, amphoterics based on alkyl imidazoline, betaines, alkylaminopropionates, alkyliminodipropionates, alkylamphoglycinates, alkylamphocarboxyglycinates, alkylamphocarboxypropinates, alkylamphopropionates, alkylamidopropylhydroxysultaines, alkyletherhydroxypropylsultaines, alkylamphopropylsulfonate, quaternary ammonium polymers, quaternary ammonium halides, polyacrylamide, polyacrylates, polyvinyl pyrrolidone, polyvinyl alcohol, alkylalcohol ethoxylates, hydroxyalkylcelluloses, alkylamidopropyl PG-dimonium chloride phosphates, alkylampho PG-glycinate phosphates, gyceryl monoalkylates, sorbitan alkylates (Spans), Pluronics, Tetronics, sodium alkyl sulfates, sodium butoxyethoxy acetate, phosphate esters, glycosides, polyglycosides, mannitol, sorbitol, polyoxyethylene alkyl ethers, grillosan, guar gum, sodium hyaluronate, polyoxyl 40 stearate, and polyoxyolkylene dimethylpolysiloxane.

However, compounds having non-hindered hydroxyl groups attached to an aromatic ring, such as ketones and alcohols, or having a mercapto group, thioether, acetamido group, or aldehyde group will typically not be compatible. Such compounds believed not compatible with trace stabilized hydrogen peroxide include: noradrenaline, adrenaline, phenylephrine hydrochloride, amethocaine, oxybuprocaine, proxymethacaine, cromolyn sodium, benoxinate hydrochloride, chloramphenicol, chlortetracycline hydrochloride, dexamethasone, dichlorphenamide, echotiophate iodide, epinephrine bitartrate, fluorometholone, gramicidin, hydrocortisone, methazolamide, natamycin, prednisolone acetate, sulfacetamide (N¹-acetylsulfanilamide), tetracycline hydrochloride and timolol maleate.

A hydrogen peroxide stabilizer, as used herein, means any of the known stabilizers of peroxy compounds including phosphonates, phosphates, stannates, etc. Physiologically compatible salts of phosphonic acids may also be used, such as diethylene triamine penta(methylene-phosphonic acid and physiologically compatible salts thereof and 1-hydroxyethylene-1,1-diphosphonic acid and physiologically acceptable salts thereof. Other stabilizers of peroxy compounds useful in the practice of the present invention are disclosed in U.S. Pat. No. 5,725,887 at, inter alia, column 5, line 55 to column 6, line 34.

The above stabilizers can be used in almost all indications previously mentioned to which the invention is applicable. However, when the solution is to come in contact with a hydrogel soft contact lens, stannate stabilizers are to be avoided as they tend to “cloud” the lens material.

When the peroxy stabilizer is diethylene triamine penta(methylene-phosphonic acid, it can be present in the solution in an amount between about 0.001 and about 0.02% by weight of the solution, or in an amount between about 0.006 and about 0.012% by weight of the solution.

When the peroxy stabilizer is 1-hydroxyethylene-1,1-diphosphonic acid it can be present in the solution in an amount between about 0.005 and about 0.2% by weight of the solution.

Stabilizers other than diethylene triamine penta(methylene-phosphonic acid and physiologically compatible salts thereof and 1-hydroxyethylene-1,1,-diphosphonic acid and physiologically acceptable salts thereof are employed in physiologically tolerable amounts.

Soluble alkaline earth metal salts can be used in the compositions and methods of the present invention in amounts between about 0.01 and 0.2% by weight of the preserved solution, or between about 0.05 and 0.1% by weight of the preserved solution. Water soluble salts of magnesium and calcium are such alkaline earth metal salts. Preserved solutions comprising about 0.05% and 0.1% alkaline earth metal salts are disclosed herein. The present inventor has discovered that addition of such soluble alkaline earth metal salts increases antifingal preservative efficacy in ophthalmic solutions preserved with low amounts of hydrogen peroxide.

The pH of the stabilized solution is between about 5.5 and about 8. Preferably, the pH of a stabilized hydrogen peroxide solution is between about 6.0 and 8.0, most preferable between about 6.5 and 7.5. The pH can be adjusted as desired by incorporation of suitable amounts of acid or base of a physiologically tolerable nature in the amounts employed, e.g. hydrochloric acid and sodium hydroxide.

There may be present in the preserved solutions according to the present invention one or more conventional, substantially inert, physiologically acceptable tonicity enhancing agents. Suitable such agents include, for example, mannitol, sorbitol, glycerol, alkali metal halides, phosphates, hydrogen phosphate, and borates, such as sodium chloride, sodium phosphate monobasic and sodium phosphate dibasic. The function of such tonicity enhanging agents is to assure approximate physiologic tonicity to the solution which is instilled in the eye or to help assure such tonicity upon dilution if dilution is necessary prior to contact with the eye due to peroxide content as indicated above.

Preferably sufficient tonicity enhancing agents are present in the solution so that it is substantially isotonic or, such that, upon decomposition or dilution of the hydrogen peroxide therein, the resulting solution is substantially isotonic, e.g. subtantially equivalent in tonicity to a 0.9% by weight aqueous sodium chloride solution.

A further optional ingredient is a thickener or viscosity enhancing agent. Any of the substances known in these categories which are ocularly acceptable can be used. Typcial suitable thickeners include, inter alia, polyvinylalcohol, hydroxy ethylcellulose, etc. Thickeners may be present in any amount up to an amount sufficient to raise the overall solution viscosity to about 1000 cps, preferably to not more than 100 cps.

In general, the stabilized hydrogen peroxide solutions of the present invention are characterized by their extraordinary stability, even under accelerated conditions, for example by heating the solutions to 100° C. for 24 hours. Thus, the shelf life of these compositions is enhanced. Moreover, the instant compositions are characterized by either physiological tolerability subsequent to hydrogen peroxide decomposition.

Another advantage in using hydrogen peroxide in ophthalmic solutions is that the trace amount of hydrogen peroxide, especially less than 100 ppm, is destroyed once comes in contact with the eye. For example, catalase existing in the eye tissue will cause the breakdown of the hydrogen peroxide into water and oxygen. As a result, the solution, upon application, becomes preservative free and greatly minimizes adverse reactions. The problems associated with other preservatives, such as the inability to break down innocuous compounds, are eliminated.

Formulation of the solutions of the invention can be made in any conventional manner. For example, all of the components other than the hydrogen peroxide and water can be placed in a container and fresh, preferably concentrated, hydrogen peroxide added thereto with mixing. Alternatively the dry components can be rubbed up with a small portion of liquid stabilizer, then the remainder of the stabilizer added, followed by the hydrogen peroxide, and most of the water. The viscosity enhancing agent, i.e. thickener, can then be added or the formed solution can be added to the thickener. One of ordinary skill in the art will be aware of numerous variations in the manner of formulating the solutions of the invention.

When it is desirable to “neutralize” the peroxide activity, any means known, such as rinsing, contacting the solution with platinum, catalase, or any other substance known to decompose hydrogen peroxide, will suffice. Additional physiological compatible peroxide neutralizing agents include reducing agent such as pyruvic acid and suitable salts thereof such as the sodium salt.

The following examples are presented for illustrative purposes and are not intended to limit the scope of this invention, but to demonstrate the stability of the peroxy solutions as stabilized in accordance with the present invention. All parts are by weight unless otherwise indicated.

### EXAMPLE 1

A solution with the following composition is prepared by admixing the following components to form a solution.

0.2% HPMC (Hydroxypropylmethylcellulose, E50LV, from Dow Chemical, USP grade)

0.27% sodium chloride

0.12% potassium chloride

0.5% boric acid

0.05% calcium chloride dihydrate

0.006% diethylenetriamine penta(methylene phosphonic acid)

0.028% sodium perborate tetrahydrate

Water QS to the volume

pH=6.8-7.0

Tonicity=220+/−15 mOsm/kg

### EXAMPLE 2

A solution with the following composition is prepared by admixing the following components to form a solution.

0.3% HPMC (Hydroxypropylmethylcellulose, E4M, from Dow Chemical, USP grade)

0.225% sodium chloride

0.1% Calcium chloride dihydrate

0.12% potassium chlofide

0.5% boric acid

0.006% diethylenetriamine penta(methylene phosphonic acid)

0.028% sodium perborate tetrahydrate

Water QS to the volume

pH=6.8-7.0

Tonicity=220+/−15 mOsm/kg

### EXAMPLE 3

A solution with the following composition is prepared by admixing the following components to form a solution.

0.3% HPMC (Hydroxypropylmethylcellulose, E4M, from Dow Chemical, USP grade)

0.263% sodium chloride

0.05% calcium chloride dihydrate

0.12% potassium chloride

0.5% boric acid

0.006% diethylenetriamine penta(methylene phosphonic acid)

0.028% sodium perborate tetrahydrate

pH=6.8-7.0

Tonicity=220+/−15 mOsm/kg

### EXAMPLE 4

Three aqueous solutions are prepared with the following compositions:

(1) 0.3% hydroxypropylmethylcellulose, 0.3% sodium chloride, 0.5% boric acid, 0.12% potassium chloride, 0.006% diethylenetriamine penta(methylene phosphonic acid), 0.028% sodium perborate, with the pH adjusted to 6.986;

(2) 0.3% hydroxypropylmethylcellulose, 0.1% calcium chloride dihydrate, 0.3% sodium chloride, 0.5% boric acid, 0.12% potassium chloride, 0.006% diethylenetriamine penta(methylene phosphonic acid), 0.028% sodium perborate, with the pH adjusted to 6.986;

(3) 0.3% hydroxypropylmethylcellulose, 0.01% calcium chloride dihydrate, 0.3% sodium chloride, 0.5% boric acid, 0.12% potassium chloride, 0.006% diethylenetriamine penta(methylene phosphonic acid), 0.028% sodium perborate, with the pH adjusted to 6.986.

5 ml of the solutions are inoculated with fungi and are assayed for fungal presence/growth at 10, 21, and 31 days after inoculation. Some growth occurs in solutions 2 and 3 between inoculation and day 10. Solution 1 shows heavy growth of fungal colonies at all time points. By day 21, however, viable fungi are not recoverable from solutions 2 and 3, nor are viable fungi recoverable from solutions 2 or 3 on day 31. Thus, the addition of calcium chloride dihydrate at concentrations of 0.01 and 0.1% effectively inhibits the growth of fungi that would otherwise be possible in a peroxide-preserved solution.

### EXAMPLE 5

Six aqueous solutions are prepared with the following compositions:

(1) 0.3% hydroxypropylmethylcellulose, 0.3% sodium chloride, 0.5% boric acid, 0.12% potassium chloride, 0.006% diethylenetriamine penta(methylene phosphonic acid), 0.028% sodium perborate, with the pH adjusted to 7;

(2) 0.3% hydroxypropylmethylcellulose, 0.03% calcium chloride dihydrate, 0.3% sodium chloride, 0.5% boric acid, 0.12% potassium chloride, 0.006% diethylenetriamine penta(methylene phosphonic acid), 0.028% sodium perborate, with the pH adjusted to 6.963;

(3) 0.3% hydroxypropylmethylcellulose, 0.2% calcium chloride dihydrate, 0.3% sodium chloride, 0.5% boric acid, 0.12% potassium chloride, 0.006. % diethylenetriamine penta(methylene phosphonic acid), 0.028% sodium perborate, with the pH adjusted to 6.981.

(4) 0.3% hydroxypropylmethylcellulose, 0.1% calcium chloride dihydrate, 0.3% sodium chloride, 0.5% boric acid, 0.12% potassium chloride, 0.006% diethylenetriamine penta(methylene phosphonic acid), 0.028% sodium perborate, with the pH adjusted to 6.94.

(5) 0.3% hydroxypropylmethylcellulose, 0.05% calcium chloride dihydrate, 0.3% sodium chloride, 0.5% boric acid, 0.12% potassium chloride, 0.006% diethylenetriamine penta(methylene phosphonic acid), 0.028% sodium perborate, with the pH adjusted to 6.972.

(6) 0.3% hydroxypropylmethylcellulose, 0.01% calcium chloride dihydrate, 0.3% sodium chloride, 0.5% boric acid, 0.12% potassium chloride, 0.006% diethylenetriamine penta(methylene phosphonic acid), 0.028% sodium perborate, with the pH adjusted to 7.006.

Growth of inoculated Cladosporium sp. is observed in these solutions as set forth in the following table. Results are from measurement of duplicate samples.

| Solution | 0 hr (Log(CFU/ml) | 14 days | 28 days | 56 days | 77 days |
|---|---|---|---|---|---|
| 1 | 4.7 | 2.7, 2.8 | 3.1, 3.1 | 3.9, 3.8 | 3.8, 3.9 |
| 2 | 4.7 | 2.2, 2.3 | 2.2, 2.2 | 3.1, 3.0 | 3.7, 3.4 |
| 3 | 4.7 | 1.7, 1.7 | 1.4, 1.5 | 1.5, 1.4 | 1.4, 1.5 |
| 4 | 4.7 | 3.1, 2.3 | 2.6, 2.0 | 2.3, 2.2 | 2.8, 2.9 |
| 5 | 4.7 | 2.3, 2.2 | 2.3, 2.3 | 2.3, 2.4 | 3.0, 2.9 |
| 6 | 4.7 | 2.5, 2.7 | 2.7, 2.8 | 3.3, 3.4 | 3.7, 3.7 |

The results demonstrate that the addition of calcium chloride dihydrate inhibits fungal growth to an extent greater than the inhibition achieved by stabilized hydrogen peroxide alone.

What is claimed is:

1. A method of inhibiting Cladosporium growth in an aqueous ophthalmic solution comprising a cellulose derivative and a hydrogen peroxide source, comprising:
providing an aqueous solution comprising a cellulose derivative and a hydrogen peroxide source, wherein said solution will support Cladosporium growth if contaminated with Cladosporium; and
admixing an effective amount of an alkaline earth metal salt with said solution to yield an alkaline earth metal-containing solution which, if contaminated with Cladosporium, will allow less Cladosporium growth than an otherwise identical solution that does not comprise an alkaline earth metal salt.

2. The method of claim 1, further comprising adjusting the pH of said alkaline earth metal-containing solution to between about 5.5 and about 8.0.

3. The method of claim 2, wherein said hydrogen peroxide source is selected from the group consisting of hydrogen peroxide, sodium perborate, sodium peroxide and urea peroxide.

4. The method of claim 3 wherein said alkaline earth metal-containing solution further comprises one or more hydrogen peroxide stabilizers selected from the group consisting of diethylene triamine penta(methylene phosphonic acid), 1-hydroxyethylidene-1,1-diphosphonic acid, and physiologically compatible salts thereof.

5. The method of claim 4 wherein said stabilizer is 1-hydroxyethylidene-1,1-diphosphonic acid or physiologically compatible salt thereof.

6. The method of claim 4 wherein said stabilizer is diethylenetriamine penta(methylene phosphonic acid).

7. The method of claim 5 wherein said solution comprises between about 0.005% and about 0.2% by weight 1-hydroxyethylidene-1,1-diphosphonic acid or physiologically compatible salt thereof.

8. The method of claim 6 wherein said solution comprises between about 0.002% and about 0.03% by weight diethylene triamine penta(methylene phosphonic acid) or a physiologically compatible salt thereof.

9. The method of claim 4, wherein said cellulose derivative is hydroxypropylmethylcellulose.

10. The method of claim 9, wherein said solution comprises between about 0.1% and 0.5% hydroxypropylmethylcellulose by weight.

11. The method of claim 10, wherein said solution comprises between about 0.05% and about 0.1% dissolved alkaline earth metal salt by weight.

12. The method of claim 11, wherein said solution comprises about 0.05% dissolved alkaline earth metal salt by weight.

13. An ophthalmic solution comprising:
(a) a hydrogen peroxide source
(b) a cellulose derivative
(c) water; and
(d) an effective amount of an alkaline earth metal salt such that if said solution is contaminated with Cladosporium, less Cladosporium growth will occur in said solution than an otherwise identical solution that does not comprise an alkaline earth metal salt.

14. The solution of claim 13, further comprising adjusting the pH of between bout 5.5 and about 8.0.

15. The solution of claim 13, wherein said hydrogen peroxide source is selected from the group consisting of hydrogen peroxide, sodium perborate, sodium peroxide and urea peroxide.

16. The solution of claim 15 wherein said one or more hydrogen peroxide stabilizers is selected from the group consisting of diethylene triamine penta(methylene phosphonic acid), 1-hydroxyethylidene-1,1-diphosphonic acid, and physiologically compatible salts thereof.

17. The solution of claim 16, wherein said cellulose derivative is hydroxypropylmethylcellulose.

18. The solution of claim 17, wherein said solution comprises between about 0.1% and 0.5% hydroxypropylmethylcellulose by weight.

19. The solution of claim 18, wherein said solution comprises between about 0.05% and about 0.1% dissolved alkaline earth metal salt by weight.

20. The aqueous solution of claim 19 that comprises, by weight, about 0.2% hydroxypropylmethylcellulose, about 0.27% sodium chloride, about 0.12% potassium chloride, about 0.5% boric acid, about 0.05% calcium chloride dihydrate, about 0.006% diethylenetriamine penta(methylene phosphonic acid), and about 0.028% sodium perborate tetrahydrate, wherein the pH of said solution is between about 6.8 and about 7.0.
